(19) United States
(12) Patent Application Publication
(10) Pub. No.: US 2004/0191834 A1
(43) Pub. Date: Sep. 30, 2004

(54) Novel method

(76) Inventors: Craig Antony Joseph Laferriere, Oakville (CA); Jan Poolman, Rixensart (BE); Moncef Mohamed Slaoui, Rixensart (BE)

Correspondence Address: GLAXOSMITHKLINE, Corporate Intellectual Property - UW2220, P.O. Box 1539, King of Prussia, PA, 19406-0939, US

(21) Appl. No.: 10/818,819

(22) Filed: Apr. 6, 2004

Related U.S. Application Data
(63) Continuation of application No. 10/111,521, filed on Aug. 2, 2002, now abandoned.
Application No. 10/111,521 is a 371 of international application No. PCT/EP00/10733, filed on Oct. 27, 2000.

(30) Foreign Application Priority Data
Oct. 28, 1999 (GB) 9925559.8

(51) Int. Cl. G01N 33/53; A61K 39/385

(52) U.S. Cl. 435/7.1

(57) ABSTRACT

The present invention relates to the field of methods of testing a vaccine response in an animal model to obtain information on the response of humans to the same vaccinogen. The present invention provides a method of determining the dose response of a human to a polysaccharide conjugate vaccine comprising an immunogenic carrier protein and a bacterial polysaccharide, said method comprising the steps of administering to an infant animal a dose amount of said conjugated vaccine, and determining the immune response of the animal to the bacterial polysaccharide as a measure of the immune response of a human. Preferred, modes of administration of vaccine in the model, dose of vaccine tested, time between doses, time of serum harvesting, method of determination of immune response, and type (and age) of infant animal used are also all provided.

### FIELD OF THE INVENTION

The present invention relates to the field of vaccine evaluation. In particular it relates to the field of methods of determining a vaccine response in an animal model to obtain information on the response of humans to the same vaccinogen.

### BACKGROUND OF THE INVENTION

The use of animals as models for the behaviour of materials (such as vaccines) administered to humans is well established.

The immune response to different dosages of T-independent antigens, polysaccharides in particular, has a phenomenon historically called high and low dose tolerance. This immunological phenomenon changes (and is made less predictable) when a polysaccharide conjugate vaccine is made (for instance by conjugating the polysaccharide antigen to a peptide or protein which provides T-helper epitopes), as there are now 2 elements to the immune response, a B-cell element related to the polysaccharide, and a T-cell element related to the carrier protein. To date, there has been no animal model able to correctly predict human immunogenicity of polysaccharide conjugate vaccines (Eby R., Koster M., Hogerman D., Malinoski F. Pneumococcal conjugate vaccines In: Vaccines. Cold Spring Harbour Laboratory Press, 1994: 119-123).

In U.S. Pat. No. 5,604,108 a method is disclosed to establish the dosage response of humans to polysaccharide conjugate vaccines by coadministering to the animal unconjugated carrier protein.

There is still a need, however, for animal models which are more predicative still of the human response. Such animal models could be advantageously used in potency tests for the lot release of batches of vaccine (to ensure that a related response in humans would be acceptable), and in pre-clinical studies to evaluate the efficacy of new formulations of conjugate without initially having to conduct human trials.

### SUMMARY OF THE INVENTION

Accordingly, the present invention provides method of determining the dose response of a human (preferably humans aged from a few days old to one year) to a polysaccharide conjugate vaccine comprising an immunogenic carrier protein and a bacterial polysaccharide, said method comprising the steps of administering to an infant animal a dose amount of said conjugated vaccine, and determining the immune response of the animal to the bacterial polysaccharide as a measure of the immune response of a human.

Preferred modes of administration of vaccine in the model, dose of vaccine tested, time between doses, time of serum harvesting, method of determination of immune response, and type (and age) of infant animal used are all provided.

Furthermore, the above model was used in order to develop a combination vaccine comprising 2 or more pneumococcal capsular polysaccharide conjugate antigens at an optimal concentration for inducing an optimal anti-polysaccharide antibody response when administered to a human. Said combinations are further aspects of this invention.

### BRIEF DESCRIPTION OF THE DRAWINGS

FIG. 1. Correlation of Opsonophagocytic Titre and [IgG] for Serotype 6B using SB Tetravalent PS-PD in Costa Rican Infant humans.

FIG. 2. Geometric Mean IgG Concentration in Costa Rican Infants 1 month after the third injection of a Tetravalent pneumococcal PS-PD vaccine (comprising serotypes 6B, 14, 19F and 23F, and aluminium phosphate adjuvant). Bars indicate the 95% confidence interval.

FIG. 3. Geometric Mean IgG Concentration in Infant Rats 1 month after the third injection of a Tetravalent pneumococcal PS-PD vaccine with aluminium phosphate adjuvant. Bars indicate the 95% confidence interval.

FIG. 4. Geometric Mean IgG Concentration in Infant Rats 2 weeks after the third injection of an 11-valent pneumococcal PS-PD vaccine also containing aluminium phosphate adjuvant. Bars indicate the 95% confidence interval.

FIG. 5. Geometric Mean IgG Concentration in Adult Rats 2 weeks after the second injection of an 11-valent pneumococcal PS-PD vaccine also containing aluminium phosphate adjuvant. Bars indicate the 95% confidence interval.

FIG. 6. Geometric Mean IgG Concentration in Infant mice (first injection when they were 2 days old) 2 weeks after the third injection of an 11-valent pneumococcal PS-PD vaccine also containing aluminium phosphate adjuvant. Bars indicate the 95% confidence interval.

FIG. 7. Geometric Mean IgG Concentration in Infant mice (first injection when they were 1 week old) 2 weeks after the third injection of an 11-valent pneumococcal PS-PD vaccine also containing aluminium phosphate adjuvant. Bars indicate the 95% confidence interval.

FIG. 8. Geometric Mean IgG Concentration in Infant mice (first injection when they were 2 weeks old) 2 weeks after the third injection of an 11-valent pneumococcal PS-PD vaccine also containing aluminium phosphate adjuvant. Bars indicate the 95% confidence interval.

FIG. 9. Geometric Mean IgG Concentration in Infant mice (first injection when they were 4 weeks old) 2 weeks after the third injection of an 11-valent pneumococcal PS-PD vaccine also containing aluminium phosphate adjuvant. Bars indicate the 95% confidence interval.

FIG. 10. Geometric Mean IgG Concentration in young mice (first injection when they were 8 weeks old) 2 weeks after the third injection of an 11-valent pneumococcal PS-PD vaccine also containing aluminium phosphate adjuvant. Bars indicate the 95% confidence interval.

FIG. 11. Geometric Mean IgG Concentration in Infant Rhesus Monkeys 1 month after the third injection of a tetravalent pneumococcal PS-PD vaccine with aluminium phosphate adjuvant. Bars indicate the 95% confidence interval.

FIG. 12. Comparisons of anti-pneumococcal PS IgG titre in infant mice (4 weeks old at first injection) injected with the pneumococcal PS conjugate vaccine sub-cutaneously (SC) vs. humans injected intra-muscularly (IM), and in infant mice (4 weeks old at first injection) injected with the pneumococcal PS conjugate vaccine intramuscularly vs. humans injected intramuscularly. The vaccine was administered 3 times with 2 weeks between injections. Serurn was collected 2 weeks after the third injection. ELISA tests were carried our as described in Examples.

### DETAILED DESCRIPTION OF THE INVENTION

According to the present invention, a new, improved method is provided in which an animal model can be used to make a predictive correlation between the dosage response in the model and the response in humans. The invention thereby provides an animal model that correctly reflects human immunogenicity to conjugate vaccines. Such a model will be extremely useful for the lot release testing of vaccines. It will also be an important preclinical research tool for designing and evaluating new vaccine compositions (formulations with new combinations of antigens, new dosages of antigens or new excipients or adjuvants) without having to carry out as many human trials as was required previously.

The present inventors have found that different animal models give different dose-response curves (the relationship between Geometric Mean Concentration of IgG antibodies against an antigen [the polysaccharide antigen in polysaccharide conjugate vaccines] and the dose of vaccine administered with a given injection schedule) with no clear indication of which animal model is predictive of human data. It was found that only infant animals showed the strong inverse dose-response (high dose tolerance) that was demonstrated later for some serotypes in human infants. In particular, the use of infant mice in such experiments matched the human infant data best given a certain correction factor—the dosage giving the maximum response in the infant mouse is at approximately 1/10 the human dosage (the dose-response curves in general also being comparable between infant mice and humans given this corrective factor).

Accordingly, one aspect of the present invention provides a method of determining the dose response (as described above) of a human to an antigen, said method comprising the steps of administering to an infant animal a dose amount of said antigen, and determining the immune response of the animal to the antigen as a measure of the immune response of a human (preferably humans from birth to 1 year).

It is envisaged that the above method and all preferred embodiments described below will be useful for any kind of antigen (e.g. protein, nucleic acid or carbohydrate —or combinations thereof). It will be particularly useful for polysaccharide conjugate vaccines comprising an immunogenic carrier protein and a bacterial polysaccharide because of the especially unpredictable nature of the immune response against these antigens as described above. Using the methods of the invention the response to the polysaccharide portion of the vaccine may be predicted. From this point onwards it should be understood that the methods involving ‘polysaccharide conjugate vaccines’ are envisaged also to be applicable to any type of antigen.

In one embodiment the inventors have found that the polysaccharide conjugate vaccine should advantageously be administered to the infant animals intramuscularly (rather than, for instance, sub-cutaneously), as the relative immunogenicity of the polysaccharide component correlates better still with data from human trials.

In a further embodiment, the dose of vaccine administered to the infant animal should range from 0.001-10 μg, and most preferably from 0.01-1 μg. Reference to amounts of polysaccharide conjugate antigen in this specification always refers to the dosage of the polysaccharide component only (independent of amount of carrier). One or more doses may be chosen from this range. The more dosage points chosen, the more information the dose response curve will yield.

Preferably, the vaccine is administered to the infant animal the same number of times as the administration protocol of the human vaccine. Therefore, for a human polysaccharide conjugate vaccine which is to be administered in a three dose schedule, when tested in the model of the invention, each animal should similarly be injected with the vaccine three times. Furthermore, if the human vaccine involves the administration of 2 doses of conjugated polysaccharide and one dose of unconjugated polysaccharide, preferably the test in the model of the invention should be carried out likewise.

Usually in human vaccines, administration is conducted with about 1-2 months (particularly 1 month) between doses (with the first injection usually administered from birth to 1 year). It has been found in the present model that the vaccine should be preferably administered to the infant animal with a time period of 1-3 weeks between doses, and a time period of approximately 2 weeks is most preferred in terms of obtaining the best predictive model for the above human administration schedule.

Preferably, a serum sample is collected from the infant animals for testing 1-4 weeks after the last dose of vaccine is administered (and most preferably after approximately 2 weeks).

A preferred method of determining the response in the animal model to the vaccine is by measuring the concentration of anti-polysaccharide antibody in the infant animal serum per dose of vaccine administered by ELISA. This can be quite accurately done if all ELISA tests are calibrated with purified antibodies (preferably monoclonal antibodies) against the polysaccharide antigen.

Preferably, the method of transposing the infant animal data into a predicted dose response in humans is by using a dose conversion factor. Infant animals (particularly infant mice) will exhibit a similar dose response curve to humans, but at approximately 1/10 of the dose for each measurement. For pre-clinical research purposes, it is envisaged that for a given concentration of anti-polysaccharide antibody in the infant animal serum per dose of vaccine administered, approximately the same anti-polysaccharide response is seen in humans (particularly from birth to 1 year) at 5-20 times (preferably about 10 times) the dose administered to the infant animal. For potency studies for vaccine lot release, human data will have been collected, and a more precise conversion factor can be ascertained from the human data available.

In the above method, the infant animals may be rats, Rhesus monkey, chinchilla, rabbits, guinea pigs or mice. The infant animals may not be humans. The time in which animals are in their infancy can vary, however in general the age of the infant animals at the time of first inoculation should be between 1 day and 12 weeks, more preferably 2 days and 8 weeks, and most preferably 2-4 weeks. For mice and rats the first inoculation should preferably be between 1 day and 6-8 weeks, more preferably between 2 days and 5 weeks, still more preferably between 2-4 weeks, and most preferably around 4 weeks old.

In a preferred embodiment, the above method is carried out with infant mice. Most preferably Balb/c infant mice. This model has been shown for the first time to be particularly suited to predicting human (preferably from birth to 1 year) dose response curves for polysaccharide conjugate vaccines. Preferably, the infant mouse is 2 days to 8 weeks old at the time of first inoculation, and most preferably about 4 weeks old at the time of first inoculation.

Any type of bacterial polysaccharide conjugates can be used in the above method—in particular where the bacterial polysaccharide component is selected from a group consisting of: a PRP capsular polysaccharide from H. influenzae type B; a capsular polysaccharide from Streptococcus pneumoniae; a capsular polysaccharide from Group B Streptococcus; a capsular polysaccharide from Group A Streptococcus; a capsular polysaccharide from meningococcus serogroup A; a capsular polysaccharide from meningococcus serogroup Y; a capsular polysaccharide from meningococcus serogroup W-135; a capsular polysaccharide from meningococcus serogroup C; and the Vi polysaccharide from Salmonella typhi. Preferably, the polysaccharide is the capsular polysaccharide from any strain of Streptococcius pneumoniae (most preferably from serotypes 1, 2, 3, 4, 5, 6B, 7F, 8, 9N, 9V, 10A, 11A, 12F, 14, 15B, 17F, 18C, 19A, 19F, 20, 22F, 23F, or 33F).

The carrier protein used to conjugate the polysaccharide to may be a peptide or a polypeptide, but should be a provider of T-helper epitopes. Any of those commonly used in the art may be used. Typical examples are diphtheria toxoid; CRM197; tetanus toxoid; inactivated or mutant pneumococcal pneumolysin; or meningococcal OMPC. Preferably the protein is H. influenzae protein D (see EP 594610-B).

A further aspect of the present invention provides a use of infant animals as a method of determining the dose response of a human (preferably from birth to 1 year) to a polysaccharide conjugate vaccine comprising an immunogenic carrier protein and a bacterial polysaccharide (particularly via the methods detailed above).

Using the above methods and uses, an optimal dose of antigen (particularly for those polysaccharide conjugates detailed above) can be ascertained for a human vaccine to induce an optimal anti-polysaccharide antibody response when administered to a human (preferably humans from birth to 1 year old). By optimal response, usually this will mean the largest antibody titres. Thus in a further aspect, the present invention provides vaccine formulations where the dose of antigen (particularly polysaccharide conjugate antigen) has been optimised by the above method.

As an example, the present inventors have revealed that for many pneumococcal polysaccharide conjugates, the immune response is not directly related to dose, but is bell-shaped—with the highest doses (usually 25 μg of polysaccharide are used in current unconjugated vaccines) and lowest doses yielding a lesser immune response than intermediate doses. For other serotypes, the dose response curve is more of a plateau in nature.

The present inventors have tested various doses of pneumococcal polysaccharide conjugate vaccines to determine which dose is optimal for a particular polysaccharide conjugate. Optimal responses for the various polysaccharide conjugates in the infant mouse model could be converted to optimal human doses. These optimal human doses were found to be at either a low, medium or high dose. A low dose is defined to be between 0.01 μg and 2.5 μg, and is preferably between 0.05 and 2 μg, and most preferably 1 μg of each conjugate (as mentioned above, reference to amounts of polysaccharide conjugate antigen refers to the dosage of the polysaccharide component only). A medium dose is defined to be between 2.5 μg and 4.5 μg, and is preferably between 2.6 and 4 μg, and most preferably 3 μg of each conjugate. A high dose is defined to be between 4.5 μg and 10 μg, and is preferably between 5 and 8 μg, and most preferably 6 μg of each conjugate.

In an eleven valent pneumococcal polysaccharide conjugate vaccine (which may be conjugated to any immunogenic protein, preferably those described above, and most preferably protein D) the following has been found:

| Serotype with optimal | Serotype with optimal | Serotype with optimal |
|---|---|---|
| (highest) immune | (highest) immune | (highest) immune |
| response at high dose | response at medium dose | response at low dose |
| 4, 9V, 14, | [1, 3]^{#}, 5, 7F, 18C | 6B, 19F, 23F |

^{#}PS 1 and 3 have optimal doses between a medium and a high dose.

The present inventors have found that by having the correct dose of conjugate in a vaccine, the resulting response can be improved up to 3 times than when the conjugate is dosed at a non-optimal level. Such increases in antibody titre can be related to larger proportions of the immunised population eliciting a protective immune response against the antigen. Properly dosed vaccines are thus highly advantageous.

A further aspect of the invention is thus a pneumococcal polysaccharide conjugate vaccine comprising one or more pneumococcal capsular polysaccharide conjugate antigens derived from serotypes 6B, 19F or 23F, and is present at a low dose in the vaccine.

Another embodiment of the invention is a pneumococcal polysaccharide conjugate vaccine comprising one or more pneumococcal capsular polysaccharide conjugate antigens derived from serotypes 1, 3, 5, 7F or 18C, and is present at a medium dose in the vaccine.

A still further embodiment of the invention is a pneumococcal polysaccharide conjugate vaccine comprising one or more pneumococcal capsular polysaccharide conjugate antigens derived from serotypes 1, 3, 4, 9V or 14, and is present at a high dose in the vaccine.

As many pneumococcal polysaccharide conjugate vaccines may be combined in a single vaccine, a combination vaccine comprising 2 or more pneumococcal capsular polysaccharide conjugate antigens at an optimal concentration for inducing an optimal anti-polysaccharide antibody response when administered to a human is also envisaged. Preferably, one or more of the pneumococcal capsular polysaccharide conjugate antigens is derived from serotypes 6B, 19F or 23F, and is present at a low dose in the vaccine. Alternatively, one or more of the pneumococcal capsular polysaccharide conjugate antigens may be derived from serotypes 1, 3, 5, 7F or 18C, and be present at a medium dose in the vaccine. Lastly, one or more of the pneumococcal capsular polysaccharide conjugate antigens may be derived from serotypes 1, 3, 4, 9V or 14, and be present at a high dose in the vaccine. Any combinations of the above 3 embodiments are also envisaged—for instance having one of each of a low dose, medium dose, and high dose polysaccharide conjugate comprised within the combination vaccine.

A particularly preferred combination vaccine comprises conjugate antigens derived from serotypes 6B, 19F and 23F present at a low dose, conjugate antigens derived from serotypes 1, 3, 5, 7F and 18C present at a medium dose, and conjugate antigens derived from serotypes 4, 9V and 14 present at a high dose in the vaccine. Alternatively, serotypes 1 and/or 3 may be present in the combination at a high dose.

A further embodiment of the invention is a vaccine comprising one of the above pneumococcal polysaccharide conjugate vaccines of the invention in combination with one or more of the following polysaccharide conjugates where the bacterial polysaccharide component is selected from a group consisting of: a PRP lo capsular polysaccharide from H. influenzae type B; a capsular polysaccharide from Group B Streptococcus; a capsular polysaccharide from Group A Streptococcus; a capsular polysaccharide from meningococcus serogroup A; a capsular polysaccharide from meningococcus serogroup Y; a capsular polysaccharide from meningococcus serogroup W-135; and a capsular polysaccharide from meningococcus serogroup C. In particular, a combination vaccine comprising the pneumococcal polysaccharide conjugate vaccine of the invention, a PRP conjugate and one or more of the aforementioned meningococcal polysaccharide conjugates is especially advantageous for use as a global vaccine against meningitis. In a preferred embodiment, most (and preferably all) of the polysaccharide conjugates are present in the vaccine at their optimal dose (as determinable by the above methods).

Preferably, the above polysaccharide conjugates are conjugated to any of the aforementioned protein carriers, preferably protein D. Although in combinations of these polysaccharide conjugates, all polysaccharides may be conjugated to the same carrier, this need not be the case. They may be individually conjugated to different carriers and combined so as to minimise possible carrier immune suppression that is sometimes observed where too much of a single carrier is used in a combination vaccine.

Preferably, the vaccines of the invention are for use in human infants (particularly from birth to 1 year).

The vaccine compositions of the invention may be formulated with an adjuvant such as alum or 3D-MPL. Preferably 3D-MPL devoid of aluminium adjuvant is used (as described in WO 00/56358). Other commonly used excipients may also be used.

The invention will now be illustrated (but not limited) by the following examples.

### EXAMPLES

### Example 1

S.pneumoniae Capsular Polysaccharide:

The tetravalent vaccine includes the capsular polysaccharides from serotypes 6B, 14, 19F and 23F. The 11-valent candidate vaccine includes the capsular polysaccharides from serotypes 1, 3, 4, 5, 6B, 7F, 9V, 14, 18C, 19F and 23F which were made essentially as described in EP 72513. Each polysaccharide is activated and derivatised using CDAP chemistry (WO 95/08348) and conjugated to the protein carrier. All the polysaccharides are conjugated in their native form, except for the serotype 3. It was reduced in molecular size.

Protein Carrier:

The protein carrier selected is the recombinant protein D (PD) from Non typeable Haemophilus influenzae, expressed in E. coli. For the Expression of protein D, the activation chemistry used to conjugate it to the above polysaccharides, and the characterisation of the conjugates, see WO 00/56360.

### Example 2

A review of the literature of pneumococcal conjugate vaccines showed it was not possible to make a clear correlation between the dosage response in animal models and the dosage response in humans. Furthermore, while different trends were reported in the literature for the various pneumococcal conjugates (bell-shaped, decreasing, and flat), the lack of statistical data made it impossible to determine if any of these were significantly different.

The net conclusion was that it was necessary to perform a dosage response study in humans with a pneumococcal conjugate vaccine (the carrier protein being protein D or “PD”).

The data from the dosage-response studies undertaken in 6 animal models with the protein D conjugate vaccine was assessed in order to observe the animal model(s) that best matched the human data from the dosage response study TetraPn005 in infant humans in Costa Rica. The assessment was done by blinding the assessors to the particular animal model. Those tested were adult and infant rat and mouse, infant Rhesus monkey, and adult chinchilla.

The results were that the infant mouse model corresponds best with the human immunogenicity data.

It is proposed that future studies will focus on young mice of about 4 weeks of age.

Human Studies Compared to Animal Studies.

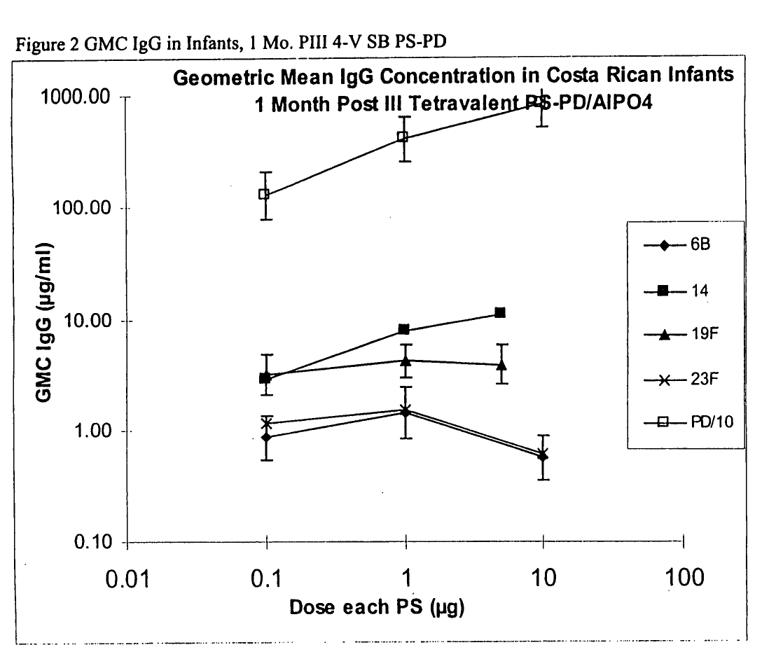

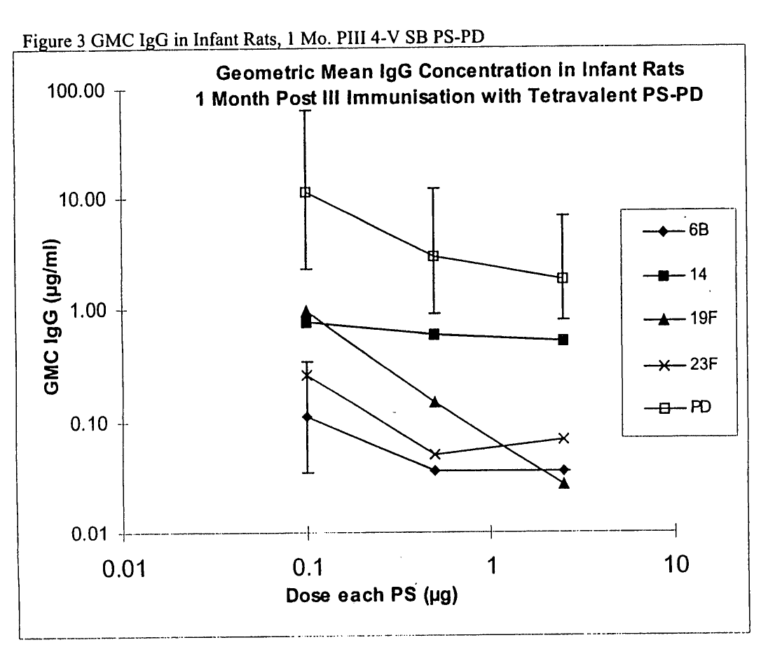

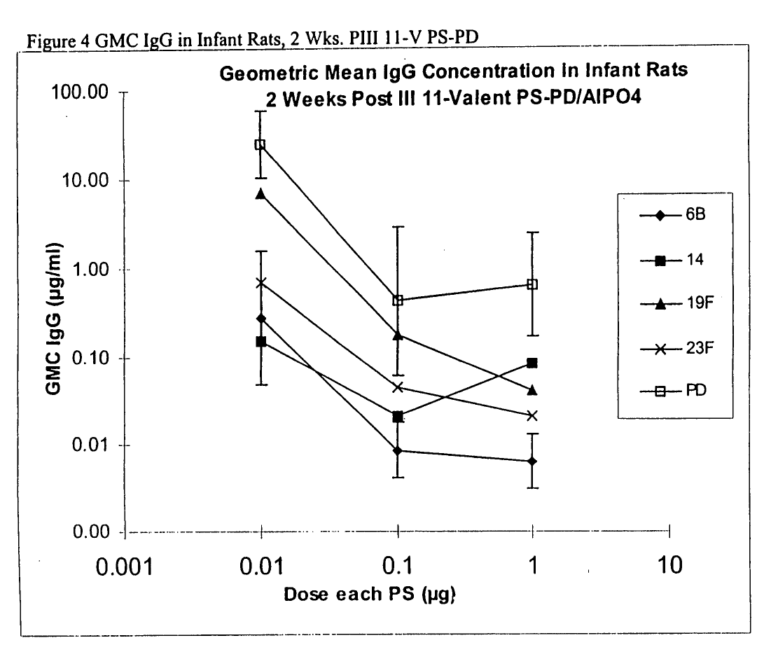

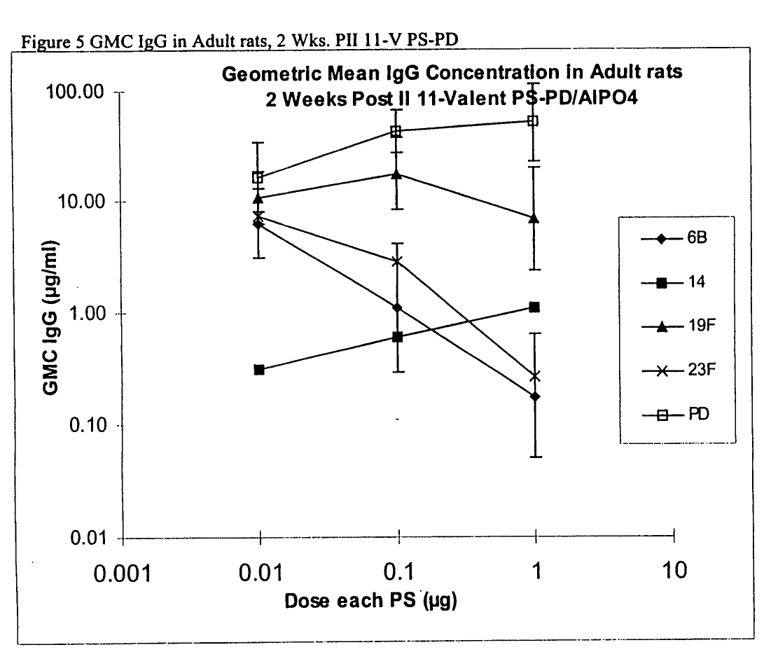

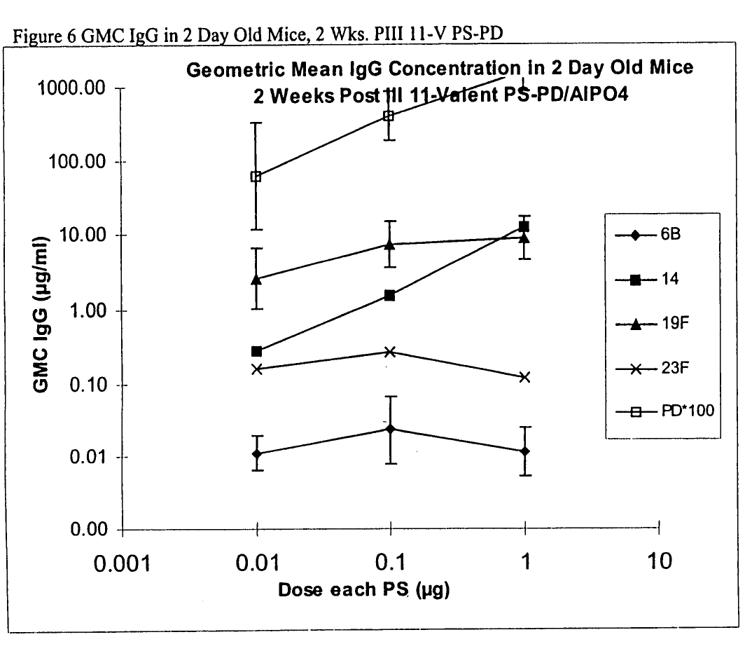

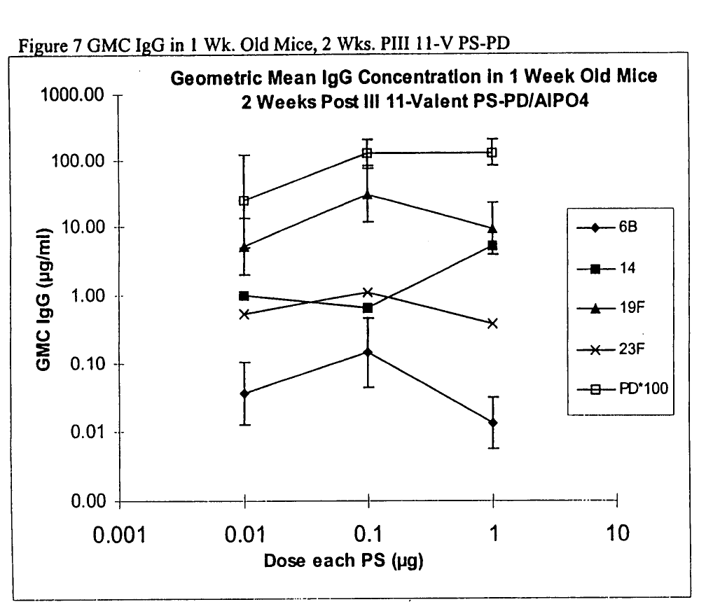

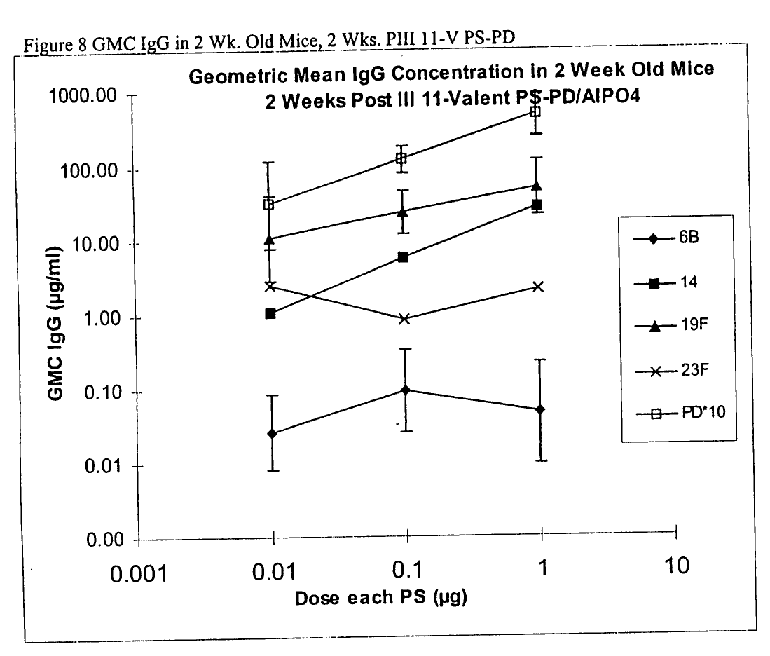

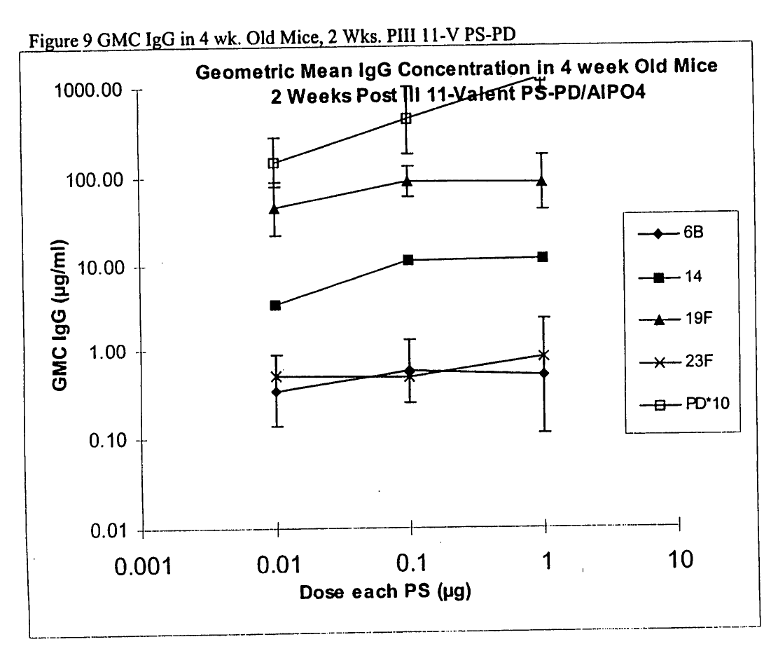

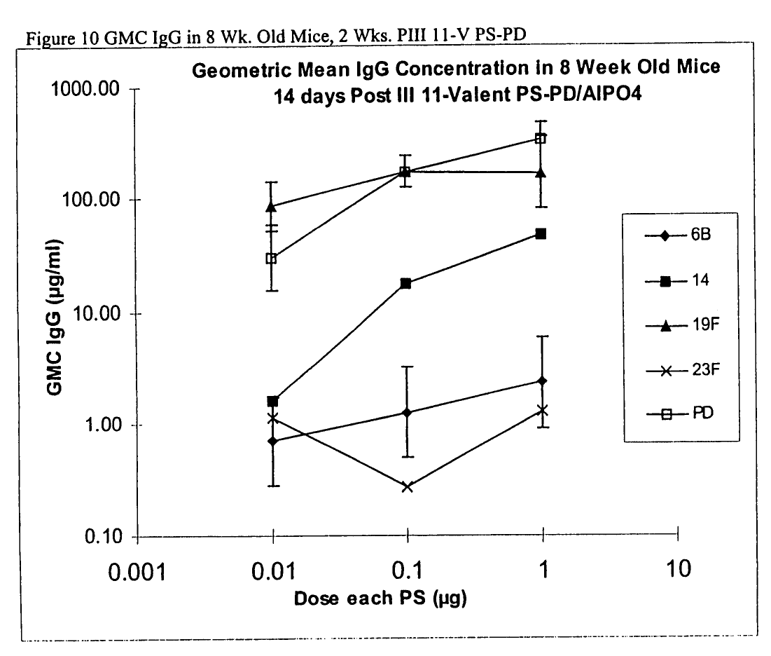

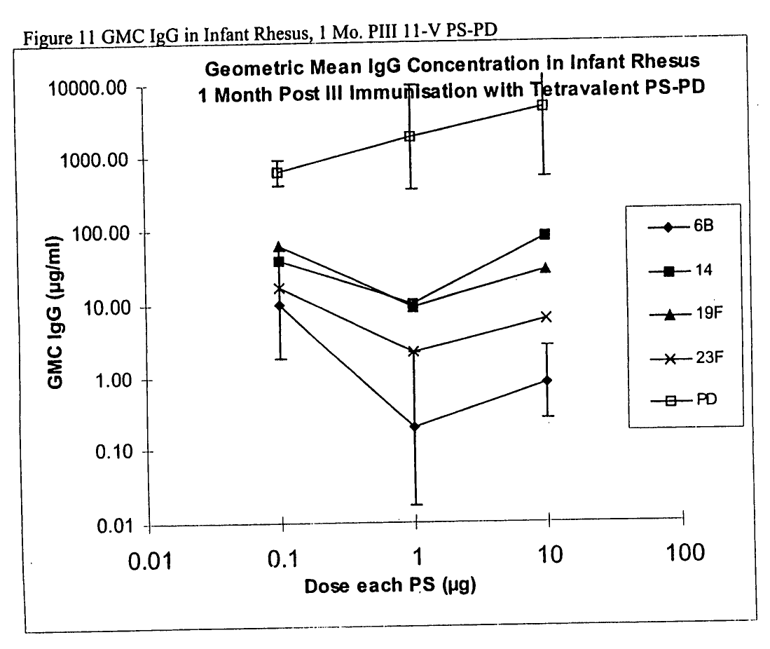

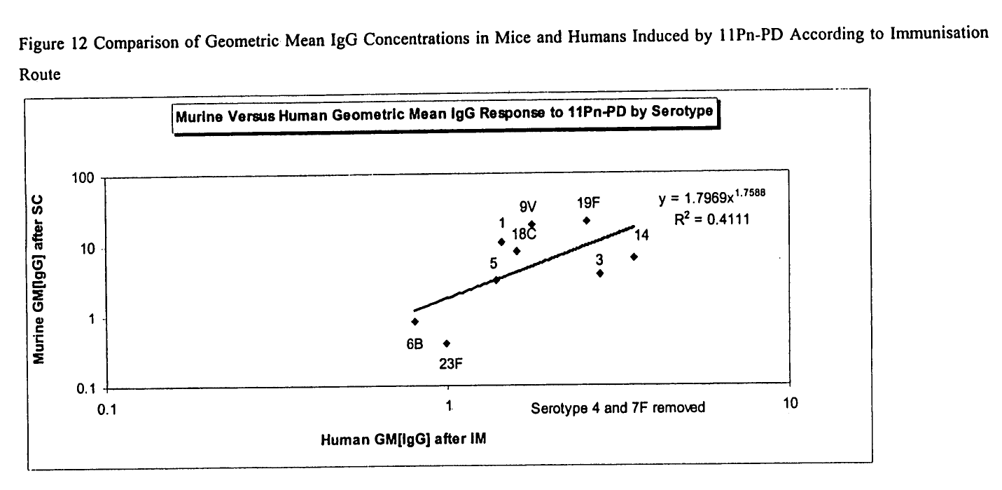

The results of dosage-response studies with a tetravalent PS-PD (pneumococcal polysaccharide—protein D) conjugate vaccine (serotypes 6B, 14, 19F and 23F) [TetraPn005] are presented in FIGS. 2, 3, and 11. Eleven valent polysaccharide vaccines were studied in FIGS. 4-10. Human data was provided by Pascal Peeters for TetraPN005 in Costa Rica.

Administration Schedule

| | Dose Schedule | Bleed date | Route |
|---|---|---|---|
| | (Age of animal | (Age of animal | (Sub-cutaneous |
| Animal Model | when injected) | when bled) | vs. Intramuscular) |
| Suckling Mouse | Day 2, 16, 30 | Day 34 | SC |
| Baby Mouse | Day 7, 21, 35 | Day 49 | SC |
| Infant Mouse | Day 14, 28, 42 | Day 56 | SC |
| Infant Mouse | Day 28, 42, 56 | Day 70 | SC |
| Young adult Mouse | Day 56, 70, 84 | Day 98 | SC |
| Infant Rats | Day 7, 21, 35 | Day 63 or 49 | SC |
| Adult Rats | Week 5, 9 | Week 11 | SC |
| Infant Rhesus | Week 8, 12, 16 | Week 20 | IM |
| Adult Chinchilla | Month 12, 13 | Month 14 | IM |
| Human | Month 3, 4, 5 | Month 6 | IM |

ELISA Data

The ELISA was performed to measure serum IgG using the WHO/CDC consensus procedure for the quantitation of IgG antibody against Streptococcus pneumoniae capsular polysaccharides. In essence, purified capsular polysaccharide is coated directly on the microtitre plate. Serum samples are pre-incubated with the cell-wall polysaccharide common to all pneumococcus and which is present in ca. 0.5% in pneumococcal polysaccharides purified according to disclosure (EP72513 B1). Jackson ImmunoLaboratories Inc. reagents were employed to detect bound IgG. The titration curves were referenced logistic log equation to internal standards (monoclonal antibodies). The calculations were performed using SoftMax Pro software. The maximum absolute error on these results expected to be within a factor of 2. The relative error is less than 30%.

Bell-Shaped Dosage-Response by Opsonophagocytosis

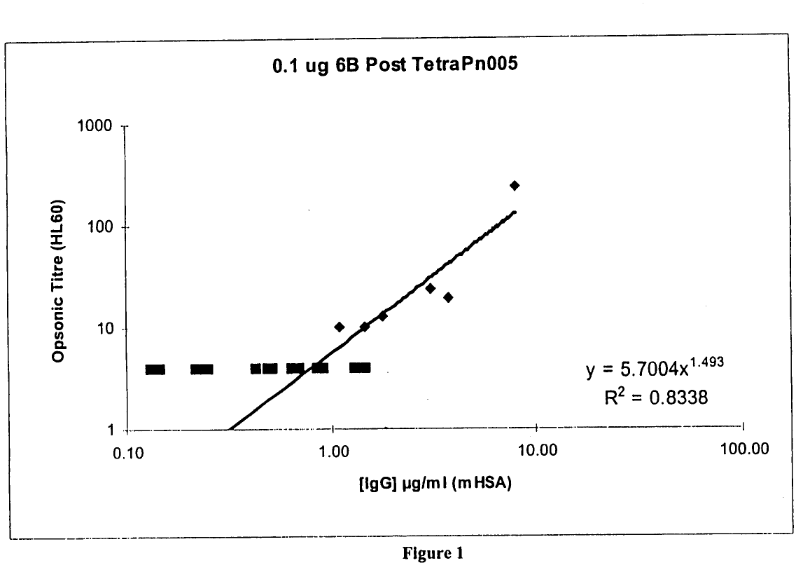

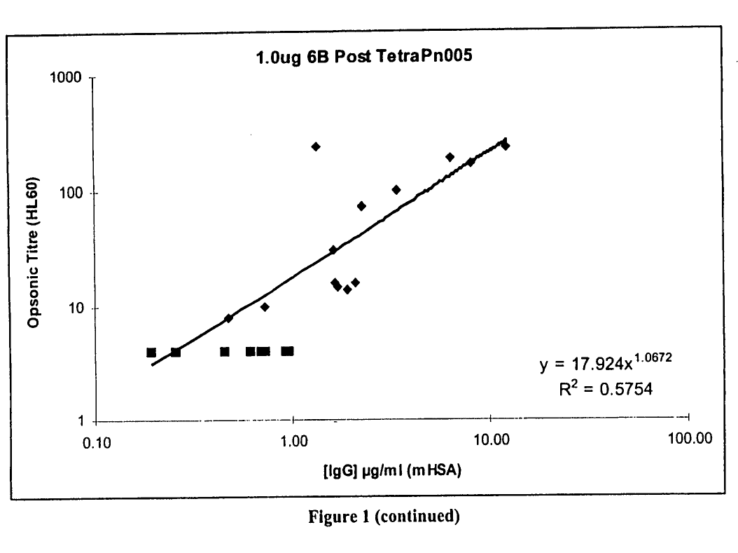

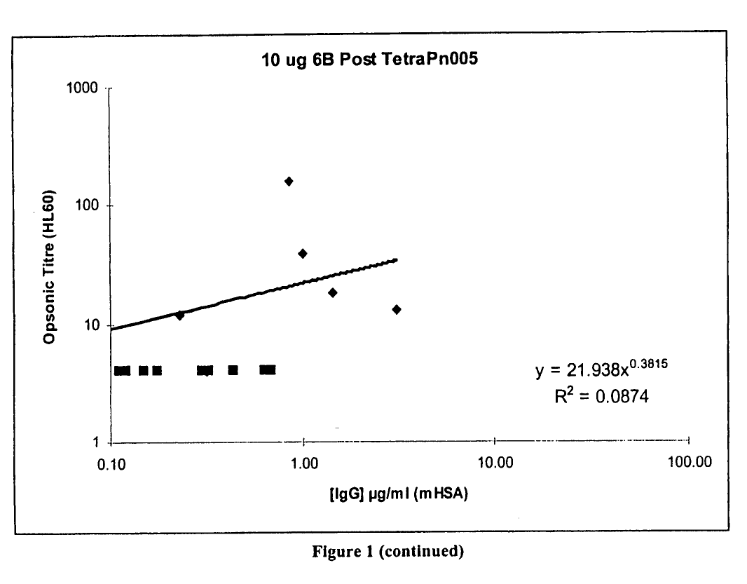

The ELISA method was adapted to use the CBER mHSA protocol (Concepcion and Frasch, Clinical and Diagnostic Laboratory Immunology (1998) 5:199) with the Costan Rican Infant Human sera. This new data provided further evidence that the 1.0 μg human dose is significantly more immunogenic than the other dosages. The geometric mean IgG concentrations for seroypte 6B are 0.50, 1.31, and 0.22 μg/ml for the 0.1, 1.0 and 10 μg dosage respectively. Interestingly, the 0.1 μg dosage has a tendency to be more immunogenic than the 10 μg dosage based on the mean of the log transformed IgG concentrations (p=0.08). These data were compared with opsonophagocytic titres determined using the CDC protocol (Romero and Steiner, Clinical and Diagnostic Laboratory Immunology (1997) 4:415). The results for serotype 6B are presented in FIG. 1 below. The seroconversion to opsonic activity are 6/24, 13/21, and 5/20 respectively (p=0.02 between the 1 and 10 μg dosage, Fisher's Exact test). The good correlation of IgG concentration with opsonophagocytic titre, and the seroconversion rate to opsonic activity, both confirm the 1.0 μg dosage as more immunogenic. However, it is interesting that even at 0.1 μg a reasonably effective vaccine formulation is obtained.

Matching Dosage Response Curves in Different Animal Systems

FIGS. 2 to 6, 10 and 11 were analysed to identify which animal model corresponded best with the human dosage response curves (FIG. 2). The animal models were blinded, but included adult and infant rats, adult and infant mice, and infant Rhesus. In addition, adult Chinchilla data were analysed. Mice of 1, 2, and 4 weeks were not included.

Although the inverse dose effect was seen generally in all infant animal models, the best results/fit was obtained for the 2 day old mouse model. This was because of the rank of the data, the bell shaped dose response being similar, and the PS 14 conjugate data showing a gradual increase in response with dose. The similarity was greater than for chinchilla (which had a very flat dosage response) and much better than that of infant rat. The similarity was also better than when infant Rhesus monkeys were used—the U-shaped response seen in infant Rhesus with the tetravalent vaccine was not observed in human infants.

Additional Studies on the Mouse Model

Balb/c mice groups were studied which were first immunised (sub-cutaneously) at 2 days old, and 1, 2, 4 and 8 weeks of age. Three immunisations took place (at day 0, 14 and 28) with 50 μl of 11-valent pneumococcal polysaccharide conjugate vaccine containing either 0.01, 0.1 or 0.5 μg per polysaccharide with 50 μg aluminium phosphate as adjuvant. A final bleed was taken at day 42. Taking into consideration the 95% confidence intervals, which may hide the true dosage-response curve, the 4 week old mouse has the best match with the human data. This is because the response to 6B is far too low to be realistic in younger mice. Note that sometime between 2 and 4 weeks of age, the mice obtain the ability to mount a higher response to 6B. This reduced immunogenicity to 6B is possibly related to the fact that antibodies to it may cross-react with double stranded DNA.

Discussion

A bell-shaped dosage-response curve has been observed with some pneumococcal polysaccharide conjugate serotypes in both humans and animal models. Other serotypes show a plateau dosage response curve. Humans under 1 year have a maximum response at approximately 1-10 μg, and infant mice have a maximum between 0.1 and 1 μg. In general the mice appear to respond in a similar way at about 1/10 the human dose.

Most of the valencies in the tetravalent pneumococcal polysaccharide-protein D conjugate vaccine shows a Bell-shaped response curve in infants (FIG. 2), and it is important to select an animal model which displays these characteristics over a reasonable dosage-range. This is important not only for the potency test, but for research purposes. In this regard, the young mouse displays the desired characteristics. The shape of the dose response in the infant mice (aged 2 days to 2-4 weeks) resembles the results of the human clinical trial shown in FIG. 2. Furthermore, for serotypes which exhibit a plateau dosage curve in humans, this is also well predicted by the infant mouse model. Since the response to 6B is too low in 2 day old mice, in addition to handling problems in the animal facilities, it is recommended that 4 week old mice be used for research purposes, and 4 or 8 week old mice be used for the potency test (8 week old mice being easier to handle still, but giving less good predictive results).

Intramuscular Versus Sub-cutaneous Administration of Vaccine

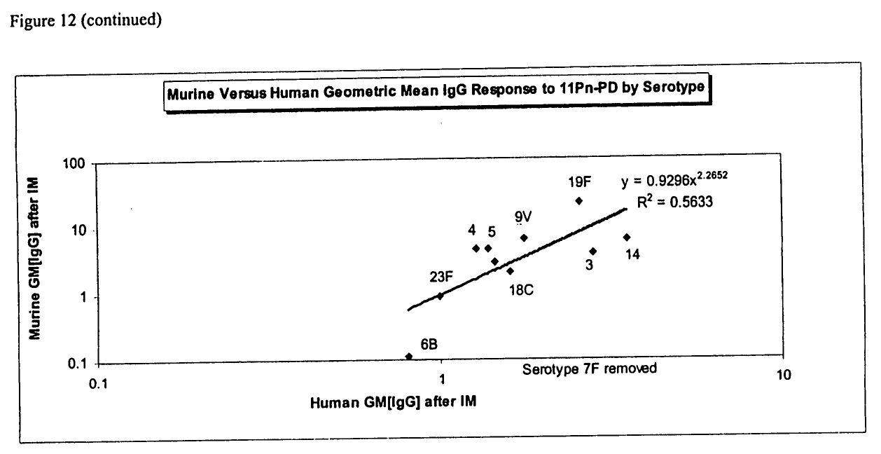

FIG. 12 shows comparisons of anti-pneumococcal PS IgG titre in infant mice (4 weeks old at first injection) injected with the 11 valent pneumococcal PS conjugate vaccine sub-cutaneously vs. humans injected intramuscularly, and in infant mice (4 weeks old at first injection) injected with the 11 valent pneumococcal PS conjugate vaccine intra-muscularly vs. humans injected intra-muscularly. The vaccine was administered 3 times with 2 weeks between injections. Serum was collected 2 weeks after the third injection. ELISA tests were carried our as described above. As can be seen, intramuscular administration of the vaccine in mice results in a further improvement to the model.

1.-26. (Cancelled)

27. A method of determining the dose response of a human to a polysaccharide conjugate vaccine comprising an immunogenic carrier protein and a bacterial polysaccharide, said method comprising the steps of administering to an infant animal a dose amount of said conjugated vaccine, and determining the immune response of the animal to the bacterial polysaccharide as a measure of the immune response of a human.

28. The method of claim 27, wherein the vaccine is administered intramuscularly to the infant animal.

29. The method of claim 27, wherein the dose of vaccine administered to the infant animal ranges from 0.001-10 μg.

30. The method of claim 29, wherein the dose of vaccine administered to the infant animal ranges from 0.01-1 μg.

31. The method of claim 27, wherein the vaccine is administered to the infant animal the same number of times as the vaccine would be administered to a human.

32. The method of claim 31, wherein the vaccine is administered three times to the infant animal.

33. The method of claim 31, wherein the vaccine is administered to the infant animal with a time period of 2 weeks between doses.

34. The method of claim 27, wherein a serum sample is collected from the infant animal for testing 2 weeks after the last dose of vaccine is administered.

35. The method of claim 27, wherein the concentration of anti-polysaccharide antibody in the infant animal serum per dose of vaccine administered is determined by ELISA.

36. The method of claim 35, wherein for a given dose of vaccine administered, approximately the same anti-polysaccharide response is seen in humans at 5-20 times the dose administered to the infant animal.

37. The method of claim 36, wherein for a given dose of vaccine administered, the same anti-polysaccharide response is seen in humans at approximately 10 times the dose administered to the infant animal.

38. The method of claim 27, wherein the infant mouse animal is an infant mouse.

39. The method of claim 38, wherein the infant mouse is 2 days to 8 weeks old at the time of first administration of the vaccine.

40. The method of claim 39, wherein the infant mouse is 4 weeks old at the time of first administration of the vaccine.

41. The method of claim 27, wherein the bacterial polysaccharide is a member selected from the group consisting of: a PRP capsular polysaccharide from H. influenzae type B; a capsular polysaccharide from Streptococcus pneumoniae; a capsular polysaccharide from Group B Streptococcus; a capsular polysaccharide from Group A Streptococcus; a capsular polysaccharide from Group A; a capsular polysaccharide from meningococcus serogroup Y; a capsular polysaccharide from menigococcus serogroup W-135; a capsular polysaccharide from meningococcus serogroup C; and the Vi polysaccharide from Salmonella typhi.

42. The method of claim 41, wherein the carrier protein is a member selected from the group consisting of: diphtheria toxoid; CRM197; tetanus toxoid; inactivated or mutant pneumococcal pneumolysin; meningococcal OMPC; and H. influenzae protein D.

43. A combination vaccine comprising 2 or more pneumococcal capsular polysaccharide conjugate antigens at an optimal concentration for inducing an optimal anti-polysaccharide antibody response when administered to a human.

44. The combination vaccine of claim 43, wherein one or more of the pneumococcal capsular polysaccharide conjugate antigens is derived from serotypes 6B, 19F or 23F, and is present at a low dose in the vaccine.

45. The combination vaccine of claim 43, wherein one or more of the pneumococcal capsular polysaccharide conjugate antigens is derived from serotypes 1, 3, 5, 7F, or 18C, and is present at a medium dose in the vaccine.

46. The combination vaccine of claim 43, wherein the one or more of the pneumococcal capsular polysaccharide conjugate antigens is derived from serotypes 4,9V or 14, and is present at a high dose in the vaccine.

47. The combination vaccine of claim 43, wherein the vaccine additionally comprises one or more further bacterial polysaccharide conjugates, said further bacterial polysaccharide being selected from the group consisting of: a PRP capsular polysaccharide from H. influenzae type B; a capsular polysaccharide from Group B Streptococcus; a capsular polysaccharide from Group A Streptococcus; a capsular polysaccharide from menigococcus serogroup A; a capsular polysaccharide from meningococcus serogroup Y; a capsular polysaccharide from meningococcus serogroup W-135; and a capsular polysaccharide from meningococcus sergroup C.

48. The combination vaccine of claim 47, wherein said vaccine comprises at least one of the further bacterial polysaccharide conjugates at an optimal concentration for inducing an optimal anti-polysaccharide antibody response when administered to a human.

49. The combination vaccine of claim 43, wherein two or more polysaccharide conjugate antigens are conjugated to one or more carrier proteins selected from the group consisting of: diphtheria toxoid; CRM197; tetanus toxoid; inactivated or mutant pneumococcal pneumolysin; menigococcal OMPC; and H. influenzae protein D.

50. A method of treating pneumococcal disease in a human host, comprising the step of administering an effective amount of the combination vaccine of claim 43, to said human host.

51. A pneumococcal polysaccharide conjugate vaccine comprising one or more pneumococcal capsular polysaccharide conjugate antigens derived from serotypes 1, 3, 5, 7F or 18C, which is present at an optimal medium dose of 2.5-4.5 μg polysaccharide per dose of vaccine.

52. The pneumococcal polysaccharide conjugate vaccine of claim 51, wherein the vaccine additionally comprises one or more pneumococcal capsular polysaccharide conjugate antigens derived from serotypes 6B, 19F or 23F, which is present at an optimal low dose of 0.01-2.5 μg polysaccharide per dose of vaccine.

53. The pneumococcal polysaccharide conjugate vaccine of claim 51, wherein the vaccine additionally comprises one or more pneumococcal capsular polysaccharide conjugate antigens derived from serotypes 4, 9V or 14, which is present at an optimal high dose of 4.5-10 μg polysaccharide per dose of vaccine.

54. The pneumococcal polysaccharide conjugate vaccine of claim 51, wherein the vaccine additionally comprises one or more further bacterial polysaccharide conjugates, said further bacterial polysaccharide being selected from a group consisting of: a PRP capsular polysaccharide from H. influenzae type B; a capsular polysaccharide from Group B Streptococcus; a capsular polysaccharide from Group A Streptococcus; a capsular polysaccharide from meningococcus serogroup A; a capsular polysaccharide from meningococcus serogroup Y; a capsular polysaccharide from meningococcus serogroup W-135; and a capsular polysaccharide from meningococcus serogroup C.

55. The pneumococcal polysaccharide conjugate vaccine of claim 54, wherein said vaccine comprises at least one of the further bacterial polysaccharide conjugates at an optimal concentration for inducing an optimal anti-polysaccharide antibody response when administered to a human.

56. The pneumococcal polysaccharide conjugate vaccine of claim 51, wherein the polysaccharide conjugate antigens are conjugated to one or more carrier proteins selected from a group consisting of: diphtheria toxoid; CRM197, tetanus toxoid; inactivated or mutant pneumococcal pneumolysin; meningococcal OMPC; and H. influenzae protein D.

57. A method of treating pneumococcal disease in a human host, comprising the step of administering the pneumococcal polysaccharide conjugate vaccine of claim 51 to said human host.

58. Use of the pneumococcal polysaccharide conjugate vaccine of claim 51 in the manufacture of a medicament for the treatment of pneumococcal disease.
